(19) United States
(12) Patent Application Publication
(10) Pub. No.: US 2026/0224492 A1
(43) Pub. Date: Aug. 6, 2026

(54) DISSOLVABLE ORALLY ADHERING TABLET

(71) Applicants: Fertin Pharma A/S, Vejle (DK); Philip Morris Products S.A., Neuchâtel (CH)

(72) Inventor: Helle Wittorff, Vejle (DK)

(73) Assignees: Fertin Pharma A/S, Vejle (DK); Philip Morris Products S.A., Neuchâtel (CH)

(21) Appl. No.: 19/151,086

(22) PCT Filed: Jan. 26, 2024
(86) PCT No.: PCT/DK2024/050013

§ 371 Date: Jul. 25, 2025

(30) Foreign Application Priority Data
Jan. 27, 2023 (EP) 23153776.2
Jul. 7, 2023 (DK) PA202370372
Jul. 7, 2023 (DK) PA202370373
Nov. 7, 2023 (DK) PA202330319

(51) Int. Cl. A61K 9/20; A61K 9/00; A61K 31/4045; A61K 31/465; A61K 31/522

(52) U.S. Cl. CPC A61K 9/2086; A61K 9/006; A61K 9/2018; A61K 31/4045; A61K 31/465; A61K 31/522

(57) ABSTRACT

A dissolvable orally adhering tablet for sustained release is disclosed, the tablet comprising an active ingredient, wherein the tablet is a multilayer tablet comprising a first layer and a second layer, the first layer being a mucoadhesive layer comprising a natural gum mucoadhesive and the second layer comprising at least one sugar alcohol.

### FIELD OF INVENTION

The invention relates to a dissolvable orally adhering tablet for sustained release according to the claims.

### BACKGROUND

Several tablets are known to provide sustained release of some active ingredients. However, such tablets may be inconvenient for the user due to the slow dissolution of the tablets. Additionally, slow dissolution may result in an unintended swallowing of active ingredients, which further would result in an unpleasant experience for the user. Besides being an unpleasant experience, unintended swallowing would prohibit buccal absorption of active ingredients. Thus, a lowered efficacy and undesired side effects such as off-taste, throat irritation and/or hiccups may be expected.

At the same time, due to the slow dissolution, providing tablets with a good mouthfeel is desirable, as users may otherwise discourage from use, or may eject the tablets prematurely due to discomfort.

It is an object of the present invention to overcome the disadvantages of the prior art.

### SUMMARY

The invention relates to a dissolvable orally adhering tablet for sustained release, the tablet comprising an active ingredient,
wherein the tablet is a multilayer tablet comprising a first layer and a second layer, the first layer being a mucoadhesive layer comprising a natural gum mucoadhesive and the second layer comprising at least one sugar alcohol.

An advantage of the invention may be that a desirable sustained delivery of active ingredients may be obtained.

Specifically, by including a natural gum mucoadhesive selectively in the first layer, the tablet may after insertion between the gum and the lip adhere to the mucosal surface of the gum or to the lip, whereby the tablet is immobilized. This immobilization facilitates sustained release, as the tablet is inserted into a low exposure region of the oral cavity with low saliva flux and minimum interaction with the tongue. Thereby the disintegration time of the tablet may be significantly prolonged. On the other hand, the adhesion either to the gum or to the lip facilitates a significant improvement in the mouthfeel for the user, as simultaneous adhesion to the gum and the lip is perceived as unpleasant, e.g. since it prevents the gum and the lip to move freely relative to each other. Based on this insight, the tablet comprises both a first layer being a mucoadhesive layer and a second layer comprising sugar alcohol.

Furthermore, by using natural gum mucoadhesive, the release profile of the active ingredient may be altered as desired.

A further advantage of the present invention may be a desirable mouthfeel and release, facilitated by the use of natural gum mucoadhesives.

A further advantage of the invention may be that the natural gum mucoadhesive upon swallowing may offer health benefits, e.g. in the bowel system due to its prebiotic properties.

It is noted that the intended use of the tablet is insertion between the gum and the lip, with the first layer facing the gum or the lip, preferably the gum, whereby the first layer adheres to the gum or lip to facilitate immobilization of the tablet. Thus, the layered structure of the tablet with the mucoadhesive layer on one side facilitates this advantageous use.

In the present context, the term “gum” refers to the gingiva, i.e. the mucosal tissue that covers the tooth.

According to an advantageous embodiment of the invention, the tablet adheres to the gum.

In a preferred embodiment according to the invention the tablet adheres to the gum. In this embodiment the first layer adheres to the gum.

It is noted within the present context that the term “dissolvable orally adhering tablet” refers to a tablet for oral administration, which is able to adhere to a mucosal surface of the oral cavity, particularly the gum or lip, preferably the gum, by means of the mucoadhesive layer. The tablet is overall dissolvable, particularly in saliva or water, in the sense that the main constituents are dissolvable in water. For the avoidance of doubt it is noted that the tablet according to the present invention is a non-chewing gum tablet, i.e. the tablet according to the present invention does not comprise gum base, and the tablet according to the present invention is not a chewing gum.

As used herein, the term “dissolve” is the process where a solid substance enters a solvent (oral saliva) to yield a solution. Unless otherwise stated, dissolving implies a full dissolving of the compound in question.

It is noted within the present context that the term “sustained release” refers to release of the active ingredient over a prolonged period of time, in particular over period of at least 30 minutes, such as at least 45 minutes, such as at least 1 hour.

It is noted within the present context that the term “mucoadhesive layer” refers to the first layer comprising natural gum mucoadhesive, whereby the first layer may adhere to a mucosal surface of the oral cavity, particularly the gum or lip, preferably the gum. It is noted that due to the tablet being inserted between the gum and the lip, the mucoadhesive effect should facilitate adhesion despite the continuously ongoing squeezing or clamping effect from the gum and the lip, such that the tablet is substantially immobilized during normal behavior of the user including e.g. eating and drinking. Thus, the term “mucoadhesive layer” and “first layer” may be used interchangeably. It is further noted that the first layer is at least partly exposed such that it is able to come into contact with an oral mucosal surface, such as the gum or lip, in order to facilitate adhesion thereto.

It is noted within the present context that the term “second layer” refers to a layer that does not provide the adhesive effect of the first layer. Thus, the second layer may act as a non-adhesive layer to allow the tablet to specifically adhere to only one side, whereby the gum and lip may move freely relative to each other.

As used herein, the term “natural gum mucoadhesive” refers to a biocompatible polysaccharide-based polymer obtained from non-marine botanical resources (i.e. non-marine plants), seaweed, or by means of bacterial fermentation, and which exhibits adhesive properties upon contact with mucosal surfaces. Thus, the natural gum mucoadhesive may facilitate active ingredient delivery and sustained release of active ingredient. Thus, it does not include synthetic polymers such as polyvinyl pyrrolidone.

Without being bound by theory it is contemplated that due to their ability to form ions, natural gums may facilitate release of active ingredients, e.g. particularly non-ionic active ingredients. It is noted that the terms “natural gum mucoadhesive” and “natural gum” may be used interchangeably.

As used herein, the term “active ingredient” refers to compounds with a physiological effect, including but not limited to nootropics, sleeping aids, antiseptics, dietary supplements, nutraceuticals, active pharmaceutical ingredients, etc. On the other hand, an active ingredient is not considered to cover e.g. sweeteners and flavors. Similarly, simple organic and inorganic acids and bases and buffers included as pH regulating agents are not considered active ingredients within the scope of the invention.

According to an advantageous embodiment of the invention, the tablet is a compressed tablet.

In the present context, the term “compressed” refers to formed by compression from a plurality of particles and/or granules, such as by means of a tableting machine, such as e.g. a rotary press. The tablet of the above embodiment is a compressed tablet formed by compression of at least a first powdered composition and a second powdered composition to give the first and second layers, respectively. Typically, the tablet may be made in a multi-step process, by compressing the first or second layer first, then sequentially compressing the other layers to obtain a multilayer tablet. When the tablet is a two-layer tablet, it may be made in a two-step process, by compressing the first or second layer, then compressing the other layer to obtain the two-layer tablet.

In an embodiment of the invention, the first layer is compressed.

In an embodiment of the invention, the second layer is compressed.

According to an advantageous embodiment of the invention, the tablet is composed of a plurality of compressed particles.

In an embodiment of the invention, first layer is composed of a plurality of compressed particles.

In an embodiment of the invention, second layer is composed of a plurality of compressed particles.

According to an advantageous embodiment of the invention, the first and the second layers are fused by compression.

In an embodiment of the invention, the active ingredient comprises one or more active ingredients selected from the group consisting of aspirin, caffeine, cannabinoid, cetirizine, chlorpheniramine maleate, dextromethorphan, dimenhydrinate, diphenhydramine, doxylamine succinate, famotidine, fexofenadine, levocetirizine, loratadine, meclizine, melatonin, omeprazole, phenylephrine, pseudoephedrine, theanine, and any combination thereof.

In an embodiment of the invention, the active ingredient is selected from the group consisting of aspirin, caffeine, cannabinoid, cetirizine, chlorpheniramine maleate, dextromethorphan, dimenhydrinate, diphenhydramine, doxylamine succinate, famotidine, fexofenadine, levocetirizine, loratadine, meclizine, melatonin, omeprazole, phenylephrine, pseudoephedrine, theanine, and any combination thereof.

In an embodiment of the invention, the active ingredient comprises one or more active pharmaceutical ingredients selected from the group consisting of diphenhydramine, cetirizine, loratadine, chlorpheniramine maleate, levocetirizine, meclizine, dextromethorphan, phenylephrine, famotidine, omeprazole, doxylamine succinate, melatonin, and any combination and mixture thereof.

In some embodiments of the invention, the active ingredient comprises diphenhydramine. In some embodiments of the invention, the active ingredient comprises cetirizine. In some embodiments of the invention, the active ingredient comprises loratadine. In some embodiments of the invention, the active ingredient comprises chlorpheniramine maleate. In some embodiments of the invention, the active ingredient comprises chlorpheniramine levocetirizine. In some embodiments of the invention, the active ingredient comprises meclizine. In some embodiments of the invention, the active ingredient comprises dextromethorphan. In some embodiments of the invention, the active ingredient comprises phenylephrine. In some embodiments of the invention, the active ingredient comprises famotidine. In some embodiments of the invention, the active ingredient comprises omeprazole. In some embodiments of the invention, the active ingredient comprises doxylamine succinate. In some embodiments of the invention, the active ingredient comprises melatonin.

In some embodiments of the invention, the active ingredient comprises one or more antihistamine ingredients selected from the group consisting of diphenhydramine, cetirizine, loratadine, chlorpheniramine maleate, levocetirizine, and any combination and mixture thereof.

In some embodiments of the invention, the active ingredient comprises one or more cough suppressant ingredients comprising dextromethorphan.

In some embodiments of the invention, the active ingredient comprises one or more motion sickness ingredients comprising meclizine.

In some embodiments of the invention, the active ingredient comprises one or more decongestant ingredients comprising phenylephrine.

In an embodiment of the invention, the active ingredient comprises one or more anti heartburn agents, such as one or more anti heartburn agents selected from the group consisting of famotidine, omeprazole, and any combination thereof.

In some embodiments of the invention, the active ingredient comprises one or more anti insomnia agents, such as one or more anti insomnia agents selected from the group consisting of doxylamine succinate, melatonin, and any combination and mixture thereof. The anti insomnia agent may also be referred to as a sleeping aid.

In an embodiment of the invention, the active ingredient comprises one or more selected from the group consisting of diphenhydramine, fexofenadine, dimenhydrinate, pseudoephedrine, aspirin, theanine, and any combination thereof.

In an embodiment of the invention, the active ingredient comprises an antihistamine, such as an antihistamine selected from the group consisting of diphenhydramine, cetirizine, fexofenadine, loratadine, chlorpheniramine maleate, levocetirizine, and any combination thereof.

In an embodiment of the invention, the active ingredient comprises an anti motion sickness agent, such as an anti motion sickness agent selected from the group consisting of dimenhydrinate, meclizine, and any combination thereof.

In an embodiment of the invention, the active ingredient comprises a cough suppressant, such as dextromethorphan.

In an embodiment of the invention, the active ingredient comprises a decongestant, such as pseudoephedrine.

In an embodiment of the invention, the active ingredient comprises an analgesic, such as aspirin.

In an embodiment of the invention, the active ingredient comprises an alertness aid, such as an alertness aid selected from the group consisting of caffeine, theanine, and any combination thereof. The terms theanine and L-theanine may be used interchangeably.

In an embodiment of the invention, the active ingredient comprises a nootropic, such as a nootropic selected from the group consisting of caffeine, theanine, creatine, bacopa, panax ginseng, Ginkgo biloba, and any combination thereof.

In an embodiment of the invention, the active ingredient comprises a dietary supplement and/or a nutraceutical.

According to an advantageous embodiment of the invention, the active ingredient comprises a buccally absorbable active ingredient

According to an advantageous embodiment of the invention, the active ingredient comprises an active pharmaceutical ingredient.

In some embodiments of the invention, the active ingredient comprises one or more active ingredients present in the oral tablet in an amount of 2-30 mg. In some embodiments of the invention, the active ingredient comprises one or more active ingredients present in the oral tablet in an amount of 2-20 mg. In some embodiments of the invention, the active ingredient comprises one or more active ingredients present in the oral tablet in an amount of 2-10 mg.

According to an advantageous embodiment of the invention, the active ingredient is selected from the group consisting of caffeine, melatonin, nicotine, and combinations of nicotine and caffeine.

According to an advantageous embodiment of the invention, the active ingredient is selected from the group consisting of caffeine, nicotine, and any combination thereof.

According to an advantageous embodiment of the invention, the active ingredient comprises melatonin.

In an embodiment of the invention, the active ingredient consists of melatonin.

According to an advantageous embodiment of the invention, the active ingredient comprises caffeine.

When the active ingredient comprises caffeine, it may be advantageous to include binder in the same layer(s) as the caffeine, especially when the caffeine is included in the second layer.

In an embodiment of the invention, the active ingredient consists of caffeine.

According to an advantageous embodiment of the invention, the active ingredient comprises nicotine.

In an embodiment of the invention, the active ingredient consists of nicotine.

In an embodiment of the invention, the active ingredient comprises nicotine and caffeine.

In an embodiment of the invention, the active ingredient comprises a nicotine salt.

In an embodiment of the invention, the active ingredient comprises nicotine bitartrate.

In an embodiment of the invention, the active ingredient comprises nicotine bound to an ion exchange resin.

In an embodiment of the invention, the active ingredient comprises nicotine bound to an ion exchange resin.

In an embodiment of the invention, the active ingredient comprises nicotine bound to polacrilex resin. As used herein, the term “NPR” refers to nicotine bound to polacrilex resin.

According to an advantageous embodiment of the invention, the tablet comprises caffeine in an amount of at least 10 mg, such as at least 20 mg.

In an embodiment of the invention, the tablet comprises caffeine in an amount of 10 to 200 mg, such as 20 to 100 mg.

In an embodiment of the invention, the tablet comprises the active ingredient in an amount of 10 to 200 mg, such as 20 to 100 mg.

According to an advantageous embodiment of the invention, the tablet comprises melatonin in an amount of at least 0.5 mg, such as at least 1 mg.

In an embodiment of the invention, the tablet comprises melatonin in an amount of 0.5 to 20 mg, such as 0.5 to 15 mg, such as 1 to 10 mg.

In an embodiment of the invention, the tablet comprises the active ingredient in an amount of 0.5 to 20 mg, such as 0.5 to 15 mg, such as 1 to 10 mg.

According to an advantageous embodiment of the invention, the tablet comprises nicotine in an amount of at least 0.5 mg, such as at least 1 mg.

In an embodiment of the invention, the tablet comprises nicotine in an amount of 0.5 to 10 mg, such as 1 to 4 mg.

In an embodiment of the invention, the tablet comprises the active ingredient in an amount of 0.5 to 10 mg, such as 1 to 4 mg.

In an embodiment of the invention, the active ingredient comprises a cannabinoid, such as one or more cannabinoids selected from the group consisting of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A), and any combination thereof. More preferably the one or more cannabinoid is or comprises CBD or THC.

In an embodiment of the invention, the tablet comprises cannabidiol (CBD). In an embodiment of the invention, the tablet comprises tetrahydrocannabiol (THC).

In an embodiment of the invention, the tablet comprises one or more cannabinoids in an amount of at least 5 mg, such as at least 10 mg, such as at least 20 mg.

In an embodiment of the invention, the tablet comprises one or more cannabinoids in an amount of 5 to 200 mg, such as 10 to 150 mg, such as 10 to 100 mg, such as 20 to 75 mg, such as 20 to 50 mg.

According to an advantageous embodiment of the invention, the active ingredient is comprised in the first layer.

An advantage of the above embodiment may be that an otherwise undesirably grittiness of the second layer may be minimized or avoided. This may be especially advantageous when including active ingredient in the second layer, particularly, when the active ingredient in the second layer has a relatively low solubility in water, e.g. caffeine.

According to an advantageous embodiment of the invention, the active ingredient is comprised in the second layer.

In an embodiment of the invention, the first layer comprises at least a part of the active ingredient.

In an embodiment of the invention, the second layer comprises at least a part of the active ingredient.

In an embodiment of the invention, the first layer comprises a part of the active ingredient, and the second layer comprises a part of the active ingredient.

In an embodiment of the invention, the active ingredient consists of a first part and a second part, the first layer comprising the first part, and the second layer comprising the second part.

According to an advantageous embodiment of the invention, the tablet comprises one or more further active ingredients.

According to an advantageous embodiment of the invention, the tablet has a dissolution time of at least 1 hour.

According to an embodiment of the invention, the tablet has a dissolution time of at least 30 minutes, such as at least 45 minutes.

According to an advantageous embodiment of the invention, the tablet has a dissolution time of no more than 10 hours, such as no more than 8 hours, such as no more than 6 hours.

In an embodiment of the invention, the tablet has a dissolution time of 1 to 10 hours, such as 2 to 8 hours, such as 3 to 6 hours.

In an embodiment of the invention, the tablet has a dissolution time of 30 minutes to 10 hours, such as 45 minutes to 8 hours, such as 1 to 6 hours.

In an embodiment of the invention, the first layer has a dissolution time of at least 30 minutes, such as at least 45 minutes, such as at least 1 hour.

In an embodiment of the invention, the first layer has a dissolution time of no more than 10 hours.

In an embodiment of the invention, the first layer has a dissolution time of 30 minutes to 10 hours, such as 45 minutes to 10 hours, such as 1 to 10 hours, such as 2 to 8 hours.

In an embodiment of the invention, the second layer has a dissolution time of at least 30 minutes, such as at least 45 minutes, such as at least 1 hour.

In an embodiment of the invention, the second layer has a dissolution time of no more than 10 hours.

In an embodiment of the invention, the second layer has a dissolution time of 30 minutes to 10 hours, such as 45 minutes to 10 hours, such as 1 to 10 hours, such as 2 to 8 hours.

In an embodiment of the invention, the dissolution time of the above embodiments is in vitro dissolution time.

In vitro dissolution time measurements are carried out in accordance to European Pharmacopeia 9.0, section 2.9.1, Disintegration of tablets and capsules.

In an embodiment of the invention, the dissolution time of the above embodiments is in vivo dissolution time.

According to an embodiment of the invention, the in vivo dissolution time was measured by at least 6 trained assessors, the trained assessors abstaining from eating and drinking at least 30 minutes before initiation of any test, the tablet was weighted and placed in the mouth, between the upper lip and the gum with the first facing the gum, where the in vivo dissolution time was registered as the time point where the tablet dissolution was substantially complete.

According to an embodiment of the invention, in vivo dissolution time was measured in accordance with example 4.

According to an advantageous embodiment of the invention, the tablet comprises a pH regulating agent, such as an alkaline pH regulating agent, such as an alkaline buffering agent.

Using a pH regulating agent, particularly an alkaline pH regulating agent, may be particularly advantageous when the active ingredient comprises nicotine.

In an embodiment of the invention, the first layer comprises a pH regulating agent.

In an embodiment of the invention, the pH regulating agent of the first layer is an alkaline pH regulating agent, such as an alkaline buffering agent.

In an embodiment of the invention, the second layer comprises a pH regulating agent.

In an embodiment of the invention, the pH regulating agent of the second layer is an alkaline pH regulating agent, such as an alkaline buffering agent.

In an embodiment of the invention, the pH regulating agent is a buffering agent, such as an alkaline buffering agent.

In an embodiment of the invention, the tablet comprises the pH regulating agent in an amount of at least 0.2% by weight of the tablet, such as at least 0.5% by weight of the tablet, such as at least 1% by weight of the tablet, such as at least 2% by weight of the tablet.

In an embodiment of the invention, the tablet comprises the pH regulating agent in an amount of 0.2 to 10% by weight of the tablet, such as 0.5 to 8% by weight of the tablet, such as 1 to 6% by weight of the tablet, such as 2 to 4% by weight of the tablet.

In an embodiment of the invention, the pH regulating agent is comprised in the first layer.

In an embodiment of the invention, the pH regulating agent is comprised in the second layer.

In an embodiment of the invention, the pH regulating agent comprises pH regulating agent selected from the group consisting of sodium carbonate, sodium bicarbonate, potassium carbonate, potassium bicarbonate, trometamol, amino acids, di-alkali hydrogen phosphate, tri-alkali phosphate, or any combination thereof.

In an embodiment of the invention, the pH regulating agent is selected from the group consisting of sodium carbonate, sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

In an embodiment of the invention, the pH regulating agent is selected from the group consisting of sodium carbonate, sodium bicarbonate, potassium carbonate, potassium bicarbonate, or any combination thereof.

In an embodiment of the invention, the pH regulating agent comprises or is sodium carbonate.

In an embodiment of the invention, the pH regulating agent comprises trometamol.

In the present context the term trometamol refers to (tris(hydroxymethyl)aminomethane), also sometimes referred to as tris buffer.

In an embodiment of the invention, the pH regulating agent comprises amino acid.

According to an advantageous embodiment of the invention, the natural gum mucoadhesive comprises a natural gum mucoadhesive selected from the group consisting of xanthan gum, konjac gum, tara gum, gellan gum, locust bean gum, gum arabic, alginate, pullulan, tragacanth gum, gum karaya, fenugreek gum, cassia gum, carrageenan, agar, alginic acid and salts thereof, and any combination thereof.

In an embodiment of the invention the natural gum mucoadhesive is selected from the group consisting of xanthan gum, konjac gum, tara gum, gellan gum, locust bean gum, gum arabic, pullulan, tragacanth gum, gum karaya, fenugreek gum, cassia gum, and any combination thereof.

In an embodiment of the invention, the natural gum comprises at least one natural gum selected from the group consisting of gum arabic, guar gum, locust bean gum, gellan gum, xanthan gum, and any combination thereof.

In an embodiment of the invention, the natural gum is selected from the group consisting of gum arabic, guar gum, locust bean gum, gellan gum, xanthan gum, and any combination thereof.

According to an advantageous embodiment of the invention, the natural gum comprises xanthan gum and/or gum arabic.

In an embodiment of the invention, the natural gum consists of xanthan gum and/or gum arabic.

According to an advantageous embodiment of the invention, the natural gum mucoadhesive comprises gum arabic.

In an embodiment of the invention, the natural gum mucoadhesive consists of gum arabic.

According to an advantageous embodiment of the invention, the natural gum mucoadhesive comprises xanthan gum.

In an embodiment of the invention, the natural gum mucoadhesive consists of xanthan gum.

According to an advantageous embodiment of the invention, the first layer comprises the natural gum mucoadhesive in an amount of at least 40% by weight of the tablet, such as at least 50% by weight of the first layer, such as at least 55% by weight of the first layer, such as at least 60% by weight of the first layer.

An advantage of the above embodiment may be that the tablet efficiently adheres to the gum.

According to an advantageous embodiment of the invention, the first layer comprises the natural gum mucoadhesive in an amount of no more than 90% by weight of the first layer, such as no more than 85% by weight of the first layer, such as no more than 80% by weight of the first layer.

An advantage of the above embodiment may be that it facilitates the eventual dissolution of the first layer. Having a sufficiently high content of natural gum mucoadhesive may be desirable to ensure adhesion of the table to the gum, however, a too high content of natural gum mucoadhesive may result in the first layer having an undesirable long dissolution time far exceeding the use time desired by the user. Also, a too high content of natural gum mucoadhesive may in some embodiments result in poor performance of the tablet with respect to delivery of the active ingredient.

In an embodiment of the invention, the first layer comprises the natural gum mucoadhesive in an amount of 40 to 90% by weight of the tablet, such as 50 to 90% by weight of the first layer, such as 55 to 85% by weight of the first layer, such as 60 to 80% by weight of the first layer.

According to an advantageous embodiment of the invention, the second layer comprises less than 20% by weight of mucoadhesive, such as less than 10% by weight of mucoadhesive, such as less than 5% by weight of mucoadhesive, such as less than 1% by weight of mucoadhesive, such as less than 0.2% by weight of mucoadhesive, such as being substantially free of mucoadhesive.

According to an advantageous embodiment of the invention, the second layer is free of mucoadhesive.

In an embodiment of the invention, the second layer comprises less than 20% by weight of natural gum mucoadhesive, such as less than 10% by weight of natural gum mucoadhesive, such as less than 5% by weight of natural gum mucoadhesive, such as less than 1% by weight of natural gum mucoadhesive, such as less than 0.2% by weight of natural gum mucoadhesive, such as being substantially free of natural gum mucoadhesive.

In an embodiment of the invention, the second layer is free of natural gum mucoadhesive.

In an embodiment of the invention, the tablet is free of polyvinyl pyrrolidone.

According to an advantageous embodiment of the invention, the first layer comprises sugar alcohol in an amount of at least 5% by weight of the first layer, such as at least 8% by weight of the first layer, such as at least 10% by weight of the first layer, such as at least 15% by weight of the first layer.

In an embodiment of the invention, the at least one sugar alcohol of the first layer comprises sugar alcohol in an amount of 5 to 50% by weight of the first layer, such as 8 to 40% by weight of the first layer, such as 10 to 30% by weight of the first layer, such as 15 to 30% by weight of the first layer.

According to an advantageous embodiment of the invention, the first layer comprises sugar alcohol in an amount of no more than 50% by weight of the first layer, such as no more than 40% by weight of the first layer, such as no more than 30% by weight of the first layer.

According to an advantageous embodiment of the invention, the second layer comprises sugar alcohol in an amount of at least 10% by weight of the second layer, such as at least 20% by weight of the second layer, such as at least 30% by weight of the second layer, such as at least 40% by weight of the second layer.

In an embodiment of the invention, the second layer comprises sugar alcohol in an amount of 10 to 90% by weight of the second layer, such as 20 to 80% by weight of the second layer, such as 30 to 70% by weight of the second layer, such as 40 to 60% by weight of the second layer.

According to an advantageous embodiment of the invention, the at least one sugar alcohol of the first layer comprises sugar alcohol selected from the group consisting of xylitol, maltitol, mannitol, erythritol, isomalt, sorbitol, lactitol and any combination thereof.

In an embodiment of the invention, the at least one sugar alcohol of the first layer is selected from the group consisting of xylitol, maltitol, mannitol, erythritol, isomalt, sorbitol, lactitol and any combination thereof.

According to an advantageous embodiment of the invention, the at least one sugar alcohol of the first layer comprises sugar alcohol selected from the group consisting of xylitol, maltitol, erythritol, isomalt, sorbitol, lactitol and any combination thereof.

In an embodiment of the invention, the at least one sugar alcohol of the first layer is selected from the group consisting of xylitol, maltitol, erythritol, isomalt, sorbitol, lactitol and any combination thereof.

In an embodiment of the invention, the at least one sugar alcohol of the first layer comprises sorbitol and/or xylitol, such as consists of sorbitol and/or xylitol.

In an embodiment of the invention, the at least one sugar alcohol of the first layer comprises sorbitol, such as consists of sorbitol.

In an embodiment of the invention, the at least one sugar alcohol of the first layer comprises xylitol, such as consists of xylitol.

According to an advantageous embodiment of the invention, the at least one sugar alcohol of the second layer comprises sugar alcohol selected from the group consisting of xylitol, maltitol, mannitol, erythritol, isomalt, sorbitol, lactitol and any combination thereof.

In an embodiment of the invention, the at least one sugar alcohol of the second layer is selected from the group consisting of xylitol, maltitol, mannitol, erythritol, isomalt, sorbitol, lactitol and any combination thereof.

According to an advantageous embodiment of the invention, the at least one sugar alcohol of the second layer comprises sugar alcohol selected from the group consisting of xylitol, maltitol, erythritol, isomalt, sorbitol, lactitol and any combination thereof.

In an embodiment of the invention, the at least one sugar alcohol of the second layer is selected from the group consisting of xylitol, maltitol, erythritol, isomalt, sorbitol, lactitol and any combination thereof.

In an embodiment of the invention, the at least one sugar alcohol of the second layer comprises sorbitol and/or xylitol, such as consists of sorbitol and/or xylitol.

In an embodiment of the invention, the at least one sugar alcohol of the second layer comprises sorbitol, such as consists of sorbitol.

In an embodiment of the invention, the at least one sugar alcohol of the second layer comprises xylitol, such as consists of xylitol.

According to an advantageous embodiment of the invention, the first layer comprises water-insoluble components in an amount of no more than 30% by weight of the first layer, such as no more than 20% by weight of the first layer, such as no more than 10% by weight of the first layer.

As used herein the term “water-soluble” refers to a relatively high water-solubility, for example a water-solubility of more than 5 gram of water-soluble composition or substance per 100 mL of water measured at 25 degrees Celsius, atmospheric pressure and pH of 7.0. Here, atmospheric pressure refers to a pressure of about 1 atmosphere, i.e. 101,325 Pascal (Pa) or a pressure within the range of 90,000 to 110,000 Pascal (Pa).

When referring to a “soluble” composition or substance, water-soluble is meant, unless otherwise stated.

Similarly, the term “water-insoluble” refers to a relatively low water-solubility, for example a water-solubility of less than 5 gram of water-soluble composition or substance per 100 mL of water measured at 25 degrees Celsius, atmospheric pressure and pH of 7.0. Here, atmospheric pressure refers to a pressure of about 1 atmosphere, i.e. 101,325 Pascal (Pa) or a pressure within the range of 90,000 to 110,000 Pascal (Pa).

When referring to a “insoluble” composition or substance, water-insoluble is meant, unless otherwise stated.

According to an advantageous embodiment of the invention, the first layer comprises one or more binders.

An advantage of the above embodiment may be that the dissolution rate of the first layer may be modified, i.e. the binder may be used as a dissolution modifier. Thereby, the dissolution rate of the first layer may be adjusted relative to the dissolution rate of the second layer, preferably by approaching similar dissolution rates of the first and second layers. Thus, in an advantageous embodiment of the invention, the dissolution time of the first layer is between half the dissolution time of the second layer and twice the dissolution time of the second layer.

Binder may advantageously be included in the first layer, whereby a desirable cohesiveness during tableting is achieved.

As used herein, the term “binder” refers to an ingredient promoting cohesiveness to the powder composition during tablet production and thereby facilitating production of layers and thereby tablets with a desirable mechanical strength. In the present context, natural gum mucoadhesives are not considered as binders. In an embodiment of the invention, the binders may be selected from cellulose and cellulose derivatives.

According to an advantageous embodiment of the invention, the first layer comprises binder in an amount of at least 1% by weight of the first layer, such as at least 2% by weight of the first layer, such as at least 4% by weight of the first layer.

According to an advantageous embodiment of the invention, the binder of the first layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

According to an advantageous embodiment of the invention, the binder of the first layer comprises at least one binder selected from the group consisting of hydroxypropyl cellulose, hydroxypropyl methylcellulose, microcrystalline cellulose, carboxymethylcellulose, and any combination thereof.

According to an advantageous embodiment of the invention, the second layer comprises binder.

An advantage of the above embodiment may be that the dissolution rate of the second layer may be modified, i.e. the binder may be used as a dissolution modifier. Thereby, the dissolution rate of the second layer may be adjusted relative to the dissolution rate of the first layer, preferably by approaching similar dissolution rates of the first and second layers. Thus, in an advantageous embodiment of the invention, the dissolution time of the first layer is between half the dissolution time of the second layer and twice the dissolution time of the second layer.

Another advantage of the above embodiment may be that the dissolution rate of the first layer and the second layer may be synchronized or at least substantially synchronized. Especially, when the second layer comprises an active ingredient, the synchronized or substantially synchronized dissolution, may provide an improved mouthfeel and taste masking.

Binder may advantageously be included in the second layer, whereby a desirable cohesiveness during tableting is achieved.

Another advantage of the above embodiment may be that a smoother surface, less gritty of the second layer may be obtained during use of the tablet, especially when the second layer comprises an active ingredient with a relatively low solubility in water, such as caffeine.

According to an advantageous embodiment of the invention, the second layer comprises binder in an amount of at least 1% by weight of the second layer, such as at least 2% by weight of the second layer, such as at least 4% by weight of the second layer.

According to an advantageous embodiment of the invention, the binder of the second layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

According to an advantageous embodiment of the invention, the binder of the second layer comprises at least one binder selected from the group consisting of hydroxypropyl cellulose, hydroxypropyl methylcellulose, microcrystalline cellulose, carboxymethyl cellulose, and any combination thereof.

According to an advantageous embodiment of the invention, the first layer constitutes at least 10% by weight of the tablet, such as at least 20% by weight of the tablet, such as at least 30% by weight of the tablet.

In an embodiment of the invention, the first layer constitutes 10 to 60% by weight of the tablet, such as 10 to 50% by weight of the tablet, such as 20 to 30% by weight of the tablet.

According to an advantageous embodiment of the invention, the second layer constitutes at least 40% by weight of the tablet, such as at least 50 by weight of the tablet, such as at least 60% by weight of the tablet.

In an embodiment of the invention, the second layer constitutes 40 to 90% by weight of the tablet, such as 50 to 90% by weight of the tablet, such as 70 to 80% by weight of the tablet.

According to an advantageous embodiment of the invention, the tablet is a two-layer tablet.

According to an advantageous embodiment of the invention, the tablet is a three-layer tablet.

In an embodiment of the invention, the tablet comprises multiple layers, such as a third layer.

According to an advantageous embodiment of the invention, the tablet has a weight of at least 100 mg, such as at least 150 mg, such as at least 200 mg.

According to an advantageous embodiment of the invention, the tablet has a weight of no more than 600 mg, such as no more than 500 mg, such as no more than 400 mg.

In an embodiment of the invention, the tablet has a weight of 100 to 600 mg, such as 150 to 500 mg, such as 200 to 400 mg, such as around 300 mg.

According to an advantageous embodiment of the invention, the first layer has a surface, at least a part of which being substantially flat or concave.

In an embodiment of the invention, the first layer has a flat surface.

In an embodiment of the invention, the first layer has a concave surface.

According to an advantageous embodiment of the invention, the first layer has a surface comprising at least one dimple.

In an embodiment of the invention, the first layer has a flat surface comprising one or more dimples.

In an embodiment of the invention, the second layer has a convex surface.

According to an advantageous embodiment of the invention, the tablet comprises flavor.

The flavor may advantageously be used as taste masking for the active ingredient.

In an embodiment of the invention, the tablet comprises flavor in an amount of at least 0.1% by weight of the tablet.

In an embodiment of the invention, the tablet comprises flavor in an amount of 0.1 to 15.0% by weight of the tablet, such as 0.1 to 10.0% by weight of the tablet, such as 0.1 to 5.0% by weight of the tablet, such as 0.2 to 3.0% by weight of the tablet.

In an embodiment of the invention, the flavor is comprised in the first and second layer, i.e. both layers comprise flavor.

In an embodiment of the invention, the first layer comprises flavor.

In an advantageous embodiment of the invention, the flavor is comprised in the first layer.

According to an advantageous embodiment of the invention, the first layer is free of flavor.

In an embodiment of the invention, the second layer comprises flavor.

In an advantageous embodiment of the invention, the flavor is comprised in the second layer.

In an embodiment of the invention, the tablet has a first ratio between content in weight percent of flavor in the first layer and the content in weight percent of active ingredient in the first layer, a second ratio between content in weight percent of flavor in the second layer and the content in weight percent of active ingredient in the second layer, where the first ratio is smaller than the second ratio.

In an embodiment of the invention, the tablet has a first ratio between content in weight percent of active ingredient in the first layer and the content in weight percent of flavor in the first layer, a second ratio between content in weight percent of active ingredient in the second layer and the content in weight percent of flavor in the second layer, where the first ratio is greater than the second ratio.

In an embodiment of the invention, the tablet comprises high intensity sweetener.

In an embodiment of the invention, the high intensity sweetener is selected from sucralose, aspartame, salts of acesulfame, such as acesulfame potassium, alitame, saccharin and its salts, cyclamic acid and its salts, glycyrrhizin, dihydrochalcones, thaumatin, monellin, stevioside, and any combination thereof.

In an embodiment of the invention, the first layer comprises high intensity sweetener.

In an embodiment of the invention, the second layer comprises high intensity sweetener.

According to an advantageous embodiment of the invention, the second layer is free of effervescent agents.

In an embodiment of the invention, the tablet is free of effervescent agents.

### DETAILED DESCRIPTION

As used herein, the term “nicotine” refers to nicotine in any form, including free base nicotine; nicotine salts; nicotine bound to a carrier, such as nicotine bound to ion exchange resins, nicotine bound to zeolites; nicotine bound to fibres or microspheres, nicotine bound to CaCO3, nicotine bound to sugar alcohol; and mixtures thereof. Bound is here to be understood as nicotine being ionically bound, adsorbed or absorbed onto the carrier, depending on the type of carrier.

When referring to nicotine amounts in milligram, the amounts are to be understood as the nicotine dose, i.e. the amounts refers to the amount of pure nicotine.

When referring to nicotine amounts in weight percent, the amounts are to be understood as the actual amount of the nicotine source in relation to the specified term, such as the first layer or the tablet. I.e. a first layer of 75 mg comprising nicotine bitartrate in an amount of 4% by weight of the first layer, refers to a first layer comprising 3 mg of nicotine bitartrate (i.e. 1 mg of pure nicotine).

Nicotine also covers nicotine not obtained from tobacco, often referred to as synthetic nicotine. Nicotine is included in the first layer. In embodiments nicotine is included in the first layer but not in the second layer.

As used herein the term “free-base nicotine” refers to non-protonated form of nicotine. Free-base nicotine may be provided as a liquid or as mixed with an amount of ion exchange resin; water-soluble compositions, such as sugar alcohols or water-soluble fibers; or water-insoluble fibers; or modified calcium carbonate. While free-base nicotine includes both free-base nicotine extracted from tobacco as well as synthetically manufactured free-base nicotine, the free-base nicotine is not provided in the form of tobacco or powdered tobacco.

As used herein, the term “nicotine salt” refers to nicotine in ionized form bound to a counterion.

As used herein, the term “NBT” refers to nicotine bitartrate and hydrates thereof.

As used herein, the term “%” and “percent” refers to percent by weight, unless otherwise is stated.

As used herein, the term “release of active ingredient” refers to the active ingredient being made bioavailable, i.e. available for absorption over the mucous membrane in the oral cavity. While some forms of active ingredient require dissolution for being bioavailable, other forms may be readily absorbed into the body without dissolution. For example, in order for the active ingredient to be bioavailable, the matrix of the tablet should be disintegrated. Some forms of active ingredient require the active ingredient to further be released from e.g. a carrier, e.g. nicotine from a nicotine-ion exchange resin such as nicotine polacrilex. Other active ingredient forms, such active ingredient salts, may readily dissolve upon disintegration of the matrix of the tablet. Still, some active ingredient forms may not require dissolving. This applies for e.g. nicotine free base, which is released upon disintegration of the solid formulation matrix.

As used herein, the term “pH regulating agent” refers to agents, which active adjust and regulates the pH value of the solution to which they have been added or are to be added. In the present context, pH regulating agents do not include active ingredients.

When referring to amounts of an ingredient by terms such as “less than”, “no more than”, this generally refers to the particular ingredient being absent or present in a range from trace amounts to the specified maximum amount.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. The flavors can be natural or synthetic flavors.

Typically, the tablet may comprise ingredients selected from the group consisting of fillers, flavors, binders, disintegrants, hereunder super disintegrants, emulsifiers, antioxidants, pH regulating agents hereunder alkaline and acidic pH regulating agents, high intensity sweeteners, colors, glidants, lubricants, or any combination thereof.

In an embodiment of the invention, the disintegrants comprise or consists of disintegrants selected from the group consisting of crospovidone, croscarmellose, sodium starch glycolate, and any combination thereof.

In an advantageous embodiment of the invention, the tablet comprises bulk sweetener as filler ingredient.

In an advantageous embodiment of the invention, the first layer comprises bulk sweetener as filler ingredient.

In an advantageous embodiment of the invention, the second layer comprises bulk sweetener as filler ingredient.

In an advantageous embodiment of the invention, the first and second layers comprise bulk sweetener as filler ingredient.

The tablet may in addition to the at least one sugar alcohol, comprise different bulk sweeteners. Bulk sweeteners include sugar sweetener and/or sugarless sweetener.

Sugar sweeteners generally include, but are not limited to saccharide-containing components, such as sucrose, dextrose, maltose, saccharose, lactose, sorbose, dextrin, trehalose, D-tagatose, dried invert sugar, fructose, levulose, galactose, and the like, alone or in combination.

Sugarless sweeteners generally include, but are not limited to sugar alcohols (also sometimes referred to as polyols) such as xylitol, maltitol, mannitol, erythritol, isomalt, sorbitol, lactitol, and combinations thereof.

Combinations of sugar and/or non-sugar sweeteners may be used in the tablet.

The bulk sweeteners may often support the flavor profile of the tablet.

In embodiment of the invention, bulk sweeteners may be supplemented with other usable fillers including as examples, magnesium and calcium carbonate, sodium sulphate, ground limestone, silicate compounds such as magnesium and aluminum silicate, kaolin and clay, aluminum oxide, silicon oxide, talc, titanium oxide, mono-, di- and tri-calcium phosphates, fibers, plant fibers, such as wheat fiber, oat fiber, pea fiber, and combinations thereof.

High intensity artificial sweetening agents can also be used in combination with the above bulk sweeteners. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (such as from about 0.02 to about 8% by weight).

In embodiments where the tablet comprises flavor, different flavors may be used.

Usable flavors including as examples almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry, kiwi, tropical punch, tutti frutti, vanilla, or any combination thereof.

According to an embodiment of the invention, flavor may be used as taste masking for the nicotine and/or taste masking of the alkaline pH regulating agent.

In an embodiment of the invention the tablet comprises glidant. Silicon dioxide may be used as a glidant. Other glidants usable for the tablet may also be used within the scope of the invention.

In an embodiment of the invention the tablet comprises lubricant. Magnesium stearate and/or sodium stearyl fumarate may be used as a lubricant. Other lubricants usable for the tablet may also be used within the scope of the invention.

### EXAMPLES

### Example 1

### Preparation of Tablets Comprising a First and a Second Layers

The composition of second layer, i.e. the layer comprising at least one sugar alcohol, is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of first layer, i.e. the mucoadhesive layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hopper of a tableting machine.

The second layer is then compressed at a compression force of about 3-8 kN, after which the first layer is fused by compression to the second layer at a compression force of about 20-30 kN. Punch used unless otherwise specified: 12.00 mm, circular, shallow concave, B tooling.

The tablets are manufactured on a lab scale machine, for example RIVA Piccola tablet press. The tablet machine is commissioned by adjusting the fill depth and compression force so the weight and hardness of tablets match the acceptance criteria. A pre-compression force could be included to avoid capping.

### Example 2: Tablet Compositions

### Example 2A: Caffeine Tablets

400 mg tablets were made each with 300 mg second layer and 100 mg first layer. The tablets were prepared according to example 1.

C1 is made as a 400 mg one layer tablet, with ingredients given as the of 1^{st} layer.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 1
Compositions of first and second layers.

| | AT1 | AT2 | AT3 | AT4 | AT5 | AT6 | C1 |
|---|---|---|---|---|---|---|---|
| Caffeine | — | — | — | 10 | 28 | — | 30 |
| dose [mg] | | | | | | | |
| Raw material | Content in weight percent of 1^{st} layer | | | | | | |
| 1^{st} layer | | | | | | | |
| Gum arabic | 65 | 65 | 65 | 65 | 65 | 20 | 65 |
| HPC | 5 | 5 | 5 | 5 | 5 | 5 | 5 |
| Xylitol | 28 | 28 | 28 | 18 | — | 73 | 20.5 |
| MgSt | 1 | 1 | 1 | 1 | 1 | 1 | 1 |
| Flavor | 1 | 1 | 1 | 1 | 1 | 1 | 1 |
| Caffeine | — | — | — | 10 | 28 | — | 7.5 |
| Total 1^{st} layer | 100 | 100 | 100 | 100 | 100 | 100 | 100 |
| Caffeine | 10 | 30 | 75 | 30 | — | 30 | — |
| dose [mg] | | | | | | | |
| Raw material | Content in weight percent of 2^{nd} layer | | | | | | |
| 2^{nd} layer | | | | | | | |
| HPC | 7.5 | 7.5 | 7.5 | 7.5 | 7.5 | 7.5 | — |
| Xylitol | 86.67 | 80 | 65 | 80 | 90 | 80 | — |
| MgSt | 0.5 | 0.5 | 0.5 | 0.5 | 0.5 | 0.5 | — |
| Flavor | 1.8 | 1.8 | 1.8 | 1.8 | 1.8 | 1.8 | — |
| HIS | 0.2 | 0.2 | 0.2 | 0.2 | 0.2 | 0.2 | — |
| Caffeine | 3.33 | 10 | 25 | 10 | — | 10 | — |
| Total 2nd layer | 100 | 100 | 100 | 100 | 100 | 100 | — |

HPC = hydroxypropyl cellulose.
HIS = high intensity sweetener.

Preferred high intensity sweeteners (HIS) may e.g. be sucralose, acesulfame potassium, and mixtures thereof. Other high intensity sweeteners, such as aspartame, salts of acesulfame, such as acesulfame potassium, alitame, saccharin and its salts, cyclamic acid and its salts, glycyrrhizin, dihydrochalcones, thaumatin, monellin, stevioside, alone or in combination, are also usable within the scope of the invention.

Fruit flavors, and mixtures thereof, menthol, peppermint, and mixtures thereof, may be used in the above formulations as flavors. Other flavors may also be used within the scope of the invention.

In the above MgSt (magnesium stearate) is used as lubricant. Other lubricants, such as sodium stearyl fumerate may also be usable within the scope of the invention.

### Examples 2B: Melatonin Tablets and Nicotine Tablets

400 mg tablets were made each with 300 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 2
Compositions of first and second layers.

| | AT11 | AT12 | AT13 | AT14 | AT15 | AT16 | AT17 |
|---|---|---|---|---|---|---|---|
| Melatonin dose | 5 | 5 | — | — | — | — | — |
| 1^{st} layer [mg] | | | | | | | |
| Nicotine dose | — | — | — | — | 0.5 | 1 | — |
| 1^{st} layer [mg] | | | | | | | |
| Raw material | Content in weight percent of 1^{st} layer | | | | | | |
| 1^{st} layer | | | | | | | |
| Gum arabic | 70 | 70 | 70 | 70 | 70 | 70 | 70 |
| HPC | 5 | 5 | 5 | 5 | 5 | 5 | 5 |
| Sorbitol | 17.8 | 17.8 | 22.8 | 22.8 | 18.3 | 16.9 | 22.8 |
| MgSt | 1 | 1 | 1 | 1 | 1 | 1 | 1 |
| Flavor | 1 | 1 | 1 | 1 | 1 | 1 | 1 |
| HIS | 0.2 | 0.2 | 0.2 | 0.2 | 0.2 | 0.2 | 0.2 |
| Melatonin | 5 | 5 | — | — | — | — | — |
| NBT | — | — | — | — | 1.5 | 2.9 | — |
| Alkaline pH | — | — | — | — | 3 | 3 | — |
| regulating agent | | | | | | | |
| Total 1st layer | 100 | 100 | 100 | 100 | 100 | 100 | 100 |
| Melatonin dose | — | 15 | 10 | 1 | — | — | — |
| 2^{nd} layer [mg] | | | | | | | |
| Nicotine dose | — | — | — | — | — | 1 | 3 |
| 2^{nd} layer [mg] | | | | | | | |
| Raw material | Content in weight percent of 2^{nd} layer | | | | | | |
| 2^{nd} layer | | | | | | | |
| HPC | 7.5 | 7.5 | 7.5 | 7.5 | 7.5 | 7.5 | 7.5 |
| Sorbitol | 89.8 | 84.8 | 86.5 | 89.6 | 89.8 | 88.8 | 84.7 |
| MgSt | 0.5 | 0.5 | 0.5 | 0.5 | 0.5 | 0.5 | 0.5 |
| Flavor | 2 | 2 | 2 | 2 | 2 | 2 | 2 |
| HIS | 0.2 | 0.2 | 0.2 | 0.2 | 0.2 | 0.2 | 0.2 |
| Melatonin | — | 5 | 3.3 | 0.3 | — | — | — |
| NBT | — | — | — | — | — | 1 | 3.1 |
| Alkaline pH | — | — | — | — | — | — | 2 |
| regulating agent | | | | | | | |
| Total 2nd layer | 100 | 100 | 100 | 100 | 100 | 100 | 100 |

NBT = nicotine bi-tartrate (nicotine content of 32.38% by weight).
HPC = hydroxypropyl cellulose.
HIS = high intensity sweetener.

Sodium carbonate is used as the alkaline pH regulating agent. Further usable alkaline pH regulating agents include sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

Alternative ingredients as described in relation to AT1-AT6 of table 1 may also be applied for AT11-AT17.

### Example 2C

300 mg tablets were made each with 200 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 3
Compositions of first and second layers.

| | AT21 | AT22 | AT23 | AT24 | AT25 | AT26 |
|---|---|---|---|---|---|---|
| Caffeine dose 1^{st} layer [mg] | 10 | 10 | 10 | 10 | 10 | 10 |
| Raw material 1^{st} layer | Content in weight percent of 1^{st} layer | | | | | |
| Gum arabic | 65 | 65 | 65 | 65 | 65 | 65 |
| HPC | 5 | 5 | 5 | 5 | 5 | 5 |
| Xylitol | 17.8 | — | — | — | 9 | — |
| Mannitol | — | 17.8 | — | — | — | — |
| Isomalt | — | — | 17.8 | — | — | — |
| Maltitol | — | — | — | 17.8 | — | — |
| Erythtritol | — | — | — | — | 8.8 | — |
| Sorbitol | — | — | — | — | — | 17.8 |
| MgSt | 1 | 1 | 1 | 1 | 1 | 1 |
| Flavor | 1 | 1 | 1 | 1 | 1 | 1 |
| HIS | 0.2 | 0.2 | 0.2 | 0.2 | 0.2 | 0.2 |
| Caffeine | 10 | 10 | 10 | 10 | 10 | 10 |
| Total 1^{st} module | 100 | 100 | 100 | 100 | 100 | 100 |
| Caffeine dose 2nd layer [mg] | 30 | 30 | 30 | 30 | 30 | 30 |
| Raw material 2^{nd} layer | Content in weight percent of 2^{nd} layer | | | | | |
| HPC | 7.5 | 7.5 | 7.5 | 7.5 | 7.5 | 7.5 |
| Xylitol | 60 | — | — | — | — | 40 |
| Mannitol | — | 60 | — | — | — | — |
| Isomalt | — | — | 60 | — | — | — |
| Maltitol | — | — | — | 60 | — | — |
| Erythtritol | — | — | — | — | 60 | — |
| Sorbitol | — | — | — | — | — | 20 |
| MgSt | 0.5 | 0.5 | 0.5 | 0.5 | 0.5 | 0.5 |
| Flavor | 2 | 2 | 2 | 2 | 2 | 2 |
| Caffeine | 30 | 30 | 30 | 30 | 30 | 30 |
| Total 2^{nd} module | 100 | 100 | 100 | 100 | 100 | 100 |

HPC = hydroxypropyl cellulose.
HIS = high intensity sweetener.

Alternative ingredients as described in relation to AT1-AT6 may also be applied for AT21-AT26.

### Example 2D

450 mg tablets were made each with 350 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 4
Compositions of first and second layers.

| | AT31 | AT32 | AT33 | AT34 | AT35 | AT36 | AT37 | AT38 | AT39 |
|---|---|---|---|---|---|---|---|---|---|
| Melatonin dose 1^{st} | 6.5 | 6.5 | 6.5 | 6.5 | 6.5 | 6.5 | 6.5 | 6.5 | 6.5 |
| layer [mg] | | | | | | | | | |
| Raw material 1^{st} | Content in weight percent of 1^{st} layer | | | | | | | | |
| layer | | | | | | | | | |
| Gum arabic | 50 | 70 | 80 | 90 | — | — | — | — | — |
| Xanthan gum | — | — | — | — | 50 | 70 | 80 | — | — |
| Locust bean gum | — | — | — | — | — | — | — | 70 | — |
| Guar gum | — | — | — | — | — | — | — | — | 70 |
| HPC | 2 | 2 | 2 | 1.5 | 2 | 2 | 2 | 2 | 2 |
| Sorbitol | 39.5 | 19.5 | 9.5 | — | 39.5 | 19.5 | 9.5 | 19.5 | 19.5 |
| MgSt | 1 | 1 | 1 | 1 | 1 | 1 | 1 | 1 | 1 |
| Flavor | 1 | 1 | 1 | 1 | 1 | 1 | 1 | 1 | 1 |
| Melatonin | 6.5 | 6.5 | 6.5 | 6.5 | 6.5 | 6.5 | 6.5 | 6.5 | 6.5 |
| Total 1^{st} module | 100 | 100 | 100 | 100 | 100 | 100 | 100 | 100 | 100 |
| Melatonin dose 2^{nd} | 3.5 | 3.5 | 3.5 | 3.5 | 3.5 | 3.5 | 3.5 | 3.5 | 3.5 |
| layer [mg] | | | | | | | | | |
| Raw material 2^{nd} | Content in weight percent of 2^{nd} layer | | | | | | | | |
| layer | | | | | | | | | |
| Gum arabic | — | 10 | — | — | — | — | — | — | — |
| HPC | 7.5 | 7.5 | 7.5 | 7.5 | 7.5 | 7.5 | 7.5 | 7.5 | 7.5 |
| Sorbitol | 89.5 | 79.5 | 89.5 | 89.5 | 89.5 | 89.5 | 89.5 | 89.5 | 89.5 |
| MgSt | 0.5 | 0.5 | 0.5 | 0.5 | 0.5 | 0.5 | 0.5 | 0.5 | 0.5 |
| Flavor | 1.5 | 1.5 | 1.5 | 1.5 | 1.5 | 1.5 | 1.5 | 1.5 | 1.5 |
| Melatonin | 1 | 1 | 1 | 1 | 1 | 1 | 1 | 1 | 1 |
| Total 2nd layer | 100 | 100 | 100 | 100 | 100 | 100 | 100 | 100 | 100 |

HPC = hydroxypropyl cellulose.

Alternative ingredients as described in relation to AT1-AT6 may also be applied for AT31-AT38.

### Example 2E

400 mg tablets were made each with 300 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 5
Compositions of first and second layers.

| | AT41 | AT42 | AT43 | AT44 | AT45 | AT46 |
|---|---|---|---|---|---|---|
| Caffeine dose 1^{st} layer [mg] | 10 | 10 | 10 | 10 | 10 | 10 |
| Raw material 1^{st} layer | Content in weight percent of 1^{st} layer | | | | | |
| Gum arabic | 65 | 65 | 65 | 65 | 65 | 65 |
| HPC | — | 5 | 10 | — | — | 5 |
| MCC | — | — | — | 5 | — | — |
| CMC | — | — | — | — | 2.5 | — |
| HPMC | — | — | — | — | 2.5 | — |
| Xylitol | 22.8 | 17.8 | 12.8 | 17.8 | 17.8 | 17.8 |
| MgSt | 1 | 1 | 1 | 1 | 1 | 1 |
| Flavor | 1 | 1 | 1 | 1 | 1 | 1 |
| HIS | 0.2 | 0.2 | 0.2 | 0.2 | 0.2 | 0.2 |
| Caffeine | 10 | 10 | 10 | 10 | 10 | 10 |
| Total 1^{st} module | 100 | 100 | 100 | 100 | 100 | 100 |
| Caffeine dose 2^{nd} layer [mg] | 60 | 60 | 60 | 60 | 60 | 60 |
| Raw material 2^{nd} layer | Content in weight percent of 2^{nd} layer | | | | | |
| HPC | 5 | 5 | — | — | — | 10 |
| CMC | — | — | — | 5 | — | — |
| HPMC | — | — | — | — | 5 | — |
| Xylitol | 72.8 | 72.8 | 77.8 | 72.8 | 72.8 | 67.8 |
| MgSt | 0.5 | 0.5 | 0.5 | 0.5 | 0.5 | 0.5 |
| Flavor | 1.5 | 1.5 | 1.5 | 1.5 | 1.5 | 1.5 |
| HIS | 0.2 | 0.2 | 0.2 | 0.2 | 0.2 | 0.2 |
| Caffeine | 20 | 20 | 20 | 20 | 20 | 20 |
| Total 2^{nd} module | 100 | 100 | 100 | 100 | 100 | 100 |

HPC = hydroxypropyl cellulose.
HIS = high intensity sweetener.

Alternative ingredients as described in relation to AT1-AT6 may also be applied for AT41-AT46.

### Example 2F

400 mg tablets were made each with 300 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 6
Compositions of first and second layers.

| | AT51 | AT52 | AT53 | AT54 | AT55 |
|---|---|---|---|---|---|
| Caffeine dose 1^{st} layer [mg] | 10 | 10 | 40 | 40 | 40 |
| Raw material 1st layer | Content in weight percent of 1st module | | | | |
| Gum arabic | 65 | 65 | 45 | 40 | 40 |
| HPC | 5 | 5 | — | 5 | 10 |
| Xylitol | 18.8 | 18.8 | 13.8 | 13.8 | 8.8 |
| MgSt | 1 | — | 1 | 1 | 1 |
| Flavor | — | 1 | — | — | — |
| HIS | 0.2 | 0.2 | 0.2 | 0.2 | 0.2 |
| Caffeine | 10 | 10 | 40 | 40 | 40 |
| Total 1^{st} module | 100 | 100 | 100 | 100 | 100 |
| Caffeine dose 2^{nd} | 60 | 60 | — | — | — |
| layer [mg] | | | | | |
| Raw material 2^{nd} module | Content in weight percent of 2^{nd} module | | | | |
| HPC | 7.5 | 7.5 | 5 | 5 | 5 |
| Xylitol | 71.8 | 72.3 | 92.8 | 92.8 | 92.8 |
| MgSt | 0.5 | — | 0.5 | 0.5 | 0.5 |
| Flavor | — | — | 1.5 | 1.5 | 1.5 |
| HIS | 0.2 | 0.2 | 0.2 | 0.2 | 0.2 |
| Caffeine | 20 | 20 | — | — | — |
| Total 2^{nd} module | 100 | 100 | 100 | 100 | 100 |

HPC = hydroxypropyl cellulose.
HIS = high intensity sweetener.

Alternative ingredients as described in relation to AT1-AT6 may also be applied for AT51-AT53.

### Example 2G

400 mg tablets were made each with 300 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 7
Compositions of first and second layers.

| | AT61 | AT62 | AT63 | AT64 | AT65 | AT66 |
|---|---|---|---|---|---|---|
| 1st layer weight | 100 | 200 | 300 | 100 | 100 | 100 |
| [mg] | | | | | | |
| 2nd layer weight | 400 | 200 | 100 | 300 | 300 | 100 |
| [mg] | | | | | | |
| Caffeine dose 1^{st} | 7.8 | 20 | 68.4 | 10 | 10 | — |
| layer [mg] | | | | | | |
| Raw material 1^{st} | Content in weight percent of 1st module | | | | | |
| layer | | | | | | |
| Gum arabic | 85 | 82.8 | 70 | 62 | 62 | 68 |
| HPC | 5 | 5 | 5 | 5 | 5 | — |
| Sorbitol | — | — | — | 20.8 | 20.8 | — |
| Xylitol | — | — | — | — | — | 29.8 |
| MgSt | 1 | 1 | 1 | 1 | 1 | 1 |
| Flavor | 1 | 1 | 1 | 1 | 1 | 1 |
| HIS | 0.2 | 0.2 | 0.2 | 0.2 | 0.2 | 0.2 |
| Caffeine | 7.8 | 10 | 22.8 | 10 | 10 | — |
| Total 1^{st} layer | 100 | 100 | 100 | 100 | 100 | 100 |
| 2nd layer weight | 400 | 200 | 100 | 300 | 300 | 100 |
| [mg] | | | | | | |
| Caffeine dose 2^{nd} | 40 | — | — | 30 | 30 | 1 |
| layer [mg] | | | | | | |
| Raw material 2^{nd} | Content in weight percent of 2^{nd} module | | | | | |
| layer | | | | | | |
| HPC | 5 | 5 | 5 | 10 | 10 | — |
| Sorbitol | 82.8 | 92.8 | 92.8 | 77.8 | — | — |
| Xylitol | — | — | — | — | 77.8 | 97.8 |
| MgSt | 0.5 | 0.5 | 0.5 | 0.5 | 0.5 | 0.5 |
| Flavor | 1.5 | 1.5 | 1.5 | 1.5 | 1.5 | 1.5 |
| HIS | 0.2 | 0.2 | 0.2 | 0.2 | 0.2 | 0.2 |
| Caffeine | 10 | — | — | 10 | 10 | — |
| Total 2^{nd} layer | 100 | 100 | 100 | 100 | 100 | 100 |

HPC = hydroxypropyl cellulose.
HIS = high intensity sweetener.

Alternative ingredients as described in relation to AT1-AT6 may also be applied for AT61-AT66.

### Example 3: Evaluation of Tablets

AT2 (comprising 30 mg caffeine), the AT12 (comprising 20 mg melatonin), and the AT17 (comprising about 9 mg NBT corresponding to 3 mg nicotine) were evaluated by a panel of 6 trained assessors.

The trained assessors abstain from eating and drinking at least 30 minutes before initiation of any test. Each trained assessor was a healthy person appointed on an objective basis according to specified requirements.

For testing the tablet was weighted and placed in the mouth, between the upper lip and the gum, with the first layer, i.e. the mucoadhesive layer, facing the gum. At specific time points, e.g. 15, 30 and 60 minutes, the content of active ingredient was measured in the remaining tablet residue, if still present. Once the desired test time was achieved, the tablet was taken out and weighed directly into a measuring glass to be used for analysis of active ingredient content. The active ingredient content was analyzed by means of standard HPLC technique after extraction into relevant buffer. Optionally, the tablets were evaluated with respect to mouth feel, taste and other sensory parameters during testing.

If dissolution of the tablet is substantially complete before the specified time point was reached, the time of dissolution was registered as the in vivo dissolution time of the tablet.

The tablet was subject to repeat measurements for each of the 6 test persons, giving a total of 12 measurements for each sample. An average of the 12 measurements was calculated and the weight % release was calculated based on the original content of active ingredient in the sample.

The evaluated tablets were evaluated to be highly suitable delivery vehicles for active ingredients, such as caffeine, melatonin and nicotine, giving desirable mouthfeel and taste profile while providing a surprising release of active ingredients.

### Example 4: In Vivo Dissolution Time

The in vivo dissolution time (as determined according to Example 3) of selected tablets were tested and is shown in below table 8.

TABLE 8
In vivo dissolution time

| Tablet | AT61 | AT62 | AT63 | AT64 | AT65 | AT66 | AT3 |
|---|---|---|---|---|---|---|---|
| In vivo dissolution | 150 | 240 | 165 | 150 | 150 | 45 | 160 |
| time [min] | | | | | | | |

As seen from table 8, the dissolution time of the tablets could be varied significantly, where the presence of binder in AT61-AT65 and AT3 resulted in a longer dissolution time than for tablet AT66 without binder.

### Example 5: Sensory Evaluation

Caffeine containing tablets AT53-AT55 (40 mg caffeine) were evaluated according to the in vivo evaluation method outlined in Example 3 with respect to bitterness and taste intensity.

Bitterness and taste intensity was each rated between 0 and 4, with 4 being most intense bitterness or taste intensity, and with 0 being no bitterness or taste detected.

TABLE 9
Bitterness and taste intensity

| Tablet | AT53 | AT54 | AT55 |
|---|---|---|---|
| Bitterness | 0 | 0 | 0 |
| Taste intensity | 4 | 4 | 4 |

Thus, tablets without flavor in the first layer were found to have high flavor intensity and without bitterness, this without being affected by varying amount of binder in the first layer.

1. A dissolvable orally adhering tablet for sustained release,
the tablet comprising an active ingredient,
wherein the tablet is a multilayer tablet comprising a first layer and a second layer,
the first layer being a mucoadhesive layer comprising a natural gum mucoadhesive and the second layer comprising at least one sugar alcohol.

2. The tablet according to claim 1, wherein the tablet adheres to the gum.

3. The tablet according to claim 1, wherein the tablet is a compressed tablet.

4-5. (canceled)

6. The tablet according to claim 1, wherein the active ingredient comprises a buccally absorbable active ingredient.

7. The tablet according to claim 1, wherein the active ingredient comprises an active pharmaceutical ingredient.

8. The tablet according to claim 1, wherein the active ingredient is selected from the group consisting of caffeine, melatonin, nicotine, and combinations of nicotine and caffeine.

9-15. (canceled)

16. The tablet according to claim 1, wherein the active ingredient is comprised in the first layer.

17. The tablet according to claim 1, wherein the active ingredient is comprised in the second layer.

18. (canceled)

19. The tablet according to claim 1, wherein the tablet has a dissolution time of at least 1 hour.

20-21. (canceled)

22. The tablet according to claim 1, wherein the natural gum mucoadhesive comprises a natural gum mucoadhesive selected from the group consisting of xanthan gum, konjac gum, tara gum, gellan gum, locust bean gum, gum arabic, alginate, pullulan, tragacanth gum, gum karaya, fenugreek gum, cassia gum, carrageenan, agar, alginic acid and salts thereof, and any combination thereof.

23. The tablet according to claim 1, wherein the natural gum mucoadhesive comprises gum arabic.

24. (canceled)

25. The tablet according to claim 1, wherein the first layer comprises the natural gum mucoadhesive in an amount of at least 40% by weight of first layer.

26. (canceled)

27. The tablet according to claim 1, wherein the second layer comprises less than 20% by weight of mucoadhesive.

28. (canceled)

29. The tablet according to claim 1, wherein the first layer comprises sugar alcohol in an amount of at least 5% by weight of the first layer.

30-31. (canceled)

32. The tablet according to claim 1, wherein the first layer comprises sugar alcohol selected from the group consisting of xylitol, maltitol, mannitol, erythritol, isomalt, sorbitol, lactitol and any combination thereof.

33-36. (canceled)

37. The tablet according to claim 1, wherein the first layer comprises one or more binders.

38. The tablet according to claim 1, wherein the first layer comprises binder in an amount of at least 1% by weight of the first layer.

39-40. (canceled)

41. The tablet according to claim 1, wherein the second layer comprises binder.

42. The tablet according to claim 1, wherein the second layer comprises binder in an amount of at least 1% by weight of the second layer.

43. The tablet according to claim 1, wherein the binder of the second layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

44. (canceled)

45. The tablet according to claim 1, wherein the first layer constitutes at least 10% by weight of the tablet.

46-48. (canceled)

49. The tablet according to claim 1, wherein the tablet has a weight of at least 100 mg.

50-52. (canceled)

53. The tablet according to claim 1, wherein the tablet comprises flavor.

54-55. (canceled)

56. The tablet according to any of claims 1-24, wherein the first layer comprises the natural gum mucoadhesive in an amount of at least 50% by weight of the first layer.
